(19) United States
(12) Patent Application Publication
(10) Pub. No.: US 2019/0216790 A1
(43) Pub. Date: Jul. 18, 2019

(54) Methods for Treating Pruritis

(71) Applicant: The Regents of the University of California, Oakland, CA (US)

(72) Inventors: Diana M. Bautista, Berkeley, CA (US); Jamie Schwendinger-Schreck, Berkeley, CA (US)

(21) Appl. No.: 16/336,424

(22) PCT Filed: Oct. 12, 2017
(86) PCT No.: PCT/US2017/056366

§ 371 Date: Mar. 25, 2019

Related U.S. Application Data
(60) Provisional application No. 62/407,921, filed on Oct. 13, 2016.

(51) Int. Cl. A61K 31/4462; A61P 17/04; A61K 9/00; A61K 31/17; A61P 17/06

(52) U.S. Cl. CPC A61K 31/4462; A61P 17/04; A61P 17/06; A61K 31/17; A61K 9/0019

(57) ABSTRACT

The present disclosure provides compositions and methods for treating chronic itch.

### CROSS-REFERENCE

This application claims the benefit of U.S. Provisional Patent Application No. 62/407,921, filed Oct. 13, 2016, which application is incorporated herein by reference in its entirety.

### INTRODUCTION

Chronic itch is a debilitating condition that is highly prevalent; however, the molecular mechanisms of chronic itch are poorly understood. Current therapies to treat chronic itch broadly target the skin barrier (e.g., with topically applied creams) or the immune system (e.g., antihistamines; steroids; immunosuppressant drugs).

There is a need in the art for compositions and methods of treating chronic itch.

### SUMMARY

The present disclosure provides compositions and methods for treating chronic itch.

### BRIEF DESCRIPTION OF THE DRAWINGS

FIG. 1A-1E depict various aspects of the role of CXCL1 in itch.

### DEFINITIONS

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a CXCR2 antagonist” includes a plurality of such antagonists and reference to “the BLT1 antagonist” includes reference to one or more BLT1 antagonists and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides a method of treating itch in an individual, the method comprising administering to the individual an effective amount of a chemokine (C-X-C motif) receptor 2 (CXCR2) antagonist. The present disclosure provides a method of treating itch in an individual, the method comprising administering to the individual an effective amount of a leuktotriene B4 receptor (BLT1) antagonist.

In some cases, an “effective amount” of a CXCR2 antagonist or a BLT1 antagonist is an amount that is effective to reduce itch (the sensation of itching) by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months. In some cases, an “effective amount” of a CXCR2 antagonist or a BLT1 antagonist is an amount that is effective to reduce the urge to scratch by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months. In some cases, an “effective amount” of a CXCR2 antagonist or a BLT1 antagonist is an amount that is effective to reduce outward manifestations of the cause of itch (e.g., hives, etc.) by at least 10%, at least 25%, at least 50%, at least 75%, at least 80%, at least 90%, or 100%, for a period of time of from about 1 hour to about 24 hours, from about 1 day to about 7 days, from about 1 week to about 4 weeks, from about 1 month to about 6 months, or more than 6 months.

### CXCR2 Antagonists

CXCR2 antagonists that are suitable for use in the present disclosure include CXCR2 inhibitors that are allosteric inhibitors of CXCR2; CXCR2 inhibitors that are competitive inhibitors of CXCR2; and the like.

CXCR2 antagonists that are suitable for use in the present disclosure include, but are not limited to, MK-7123 (navarixin); danirixin; AZD5069; reparixin; CX4338; AZD-4721; SB-33235; elubrixin; PS-291822; SB-225002; SX-517; NSC 157449; and the like.

In some cases, the CXCR2 antagonist is MK-7123. MK-7123 (also known as navarixin; (R)-2-hydroxy-N,N-dimethyl-3-((2-((1-(5-methylfuran-2-yl)propyl)amino)-3,4-dioxocyclobut-1-en-1-yl)amino)benzamide) has the following structure:

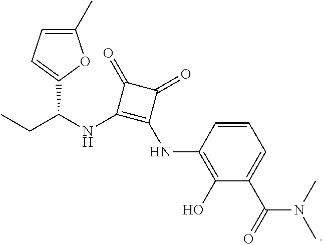 [SMILES: CC[C@@H](Nc1c(Nc2cccc(C(=O)N(C)C)c2O)c(=O)c1=O)c1ccc(C)o1]

In some cases, the CXCR2 antagonist is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl)urea. See, e.g., Miller et al. Eur J Drug Metab Pharmacokinet (2014) 39:173-181; and Miller et al. BMC Pharmacology and Toxicology (2015), 16:18. Danirixin has the following structure:

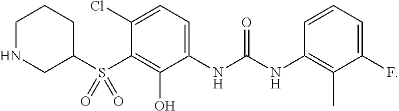 [SMILES: Cc1c(F)cccc1NC(=O)Nc1ccc(Cl)c(S(=O)(=O)C2CCCNC2)c1O]

In some cases, the CXCR2 antagonist is reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2. See, e.g., Zarbock et al. British Journal of Pharmacology (2008), 1-8. Reparixin has the following structure:

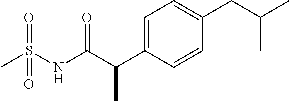 [SMILES: CC(C)Cc1ccc([C@@H](C)C(=O)NS(C)(=O)=O)cc1]

In some cases, the CXCR2 antagonist is AZD-5069. AZD-5069 has the following structure:

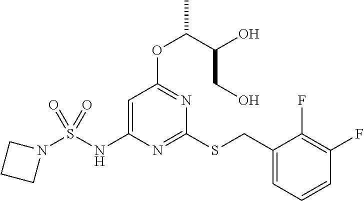 [SMILES: C[C@@H](Oc1cc(NS(=O)(=O)N2CCC2)nc(SCc2cccc(F)c2F)n1)[C@@H](O)CO]

In some cases, the CXCR2 antagonist is a compound of Formula I:

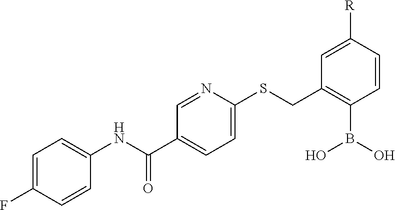

where R is H or OCF₃. In some cases, the CXCR2 antagonist is a compound of Formula I, where R is H; this compound is known as SX-517. In some cases, the CXCR2 antagonist is a compound of Formula I, where R is OCF₃.

In some cases, the CXCR2 antagonist is a compound of the following structure:

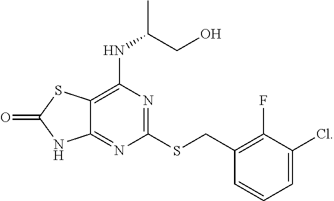 [SMILES: C[C@H](CO)Nc1nc(SCc2cccc(Cl)c2F)nc2[nH]c(=O)sc12]

In some cases, the CXCR2 antagonist is SB-225002 (N-(2-bromophenyl)-N′-(2-hydroxy-4-nitrophenyl)urea), which has the following structure:

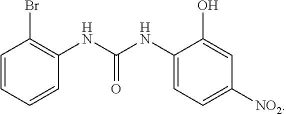 [SMILES: O=C(Nc1ccc([N+](=O)[O-])cc1O)Nc1ccccc1Br]

In some cases, the CXCR2 antagonist is elubrixin, which has the following structure:

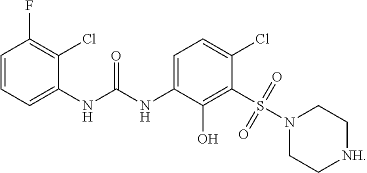 [SMILES: O=C(Nc1ccc(Cl)c(S(=O)(=O)N2CCNCC2)c1O)Nc1cccc(F)c1Cl]

In some cases, the CXCR2 antagonist is NSC 157449, which is 1-(2-hydroxy-4-nitrophenyl)-3-phenylurea.

### BLT1 Antagonists

BLT1 antagonists that are suitable for use in the present disclosure include, but are not limited to, pranlukast hydrate, montelukast, zafirlukast, MCC-847, KCA-757, CS-615, YM-158, L-740515, CP-195494, LM-1484, RS-635, A-93178, S-36496, BIIL-284, ONO-4057, BIIL-260, BIIL-315, and the like.

In some cases, the BLT1 antagonist is BIIL-284, which is a prodrug of the structure:

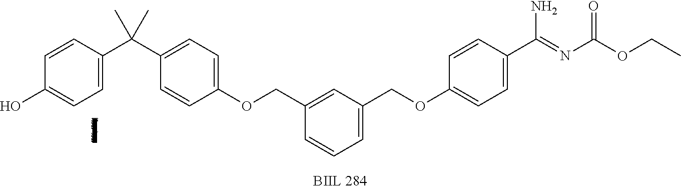 [SMILES: CCOC(=O)/N=C(\N)c1ccc(OCc2cccc(COc3ccc(C(C)(C)c4ccc(O)cc4)cc3)c2)cc1]

In some cases, the BLT1 antagonist is BIIL-260, which has the following structure:

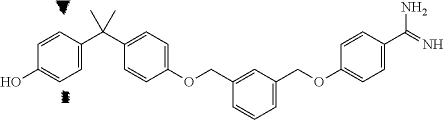 [SMILES: CC(C)(c1ccc(O)cc1)c1ccc(OCc2cccc(COc3ccc(C(=N)N)cc3)c2)cc1]

In some cases, the BLT1 antagonist is BIIL-315, which has the following structure:

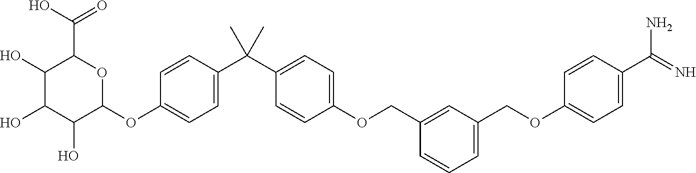 [SMILES: CC(C)(c1ccc(OCc2cccc(COc3ccc(C(=N)N)cc3)c2)cc1)c1ccc(OC2OC(C(=O)O)C(O)C(O)C2O)cc1]

In some cases, the BLT1 antagonist is U-75302 ((5S)-6-[6-[(1E,3R,5Z)-3-hydroxyundeca-1,5-dienyl]pyridin-2-yl]hexane-1,5-diol), which has the following structure:

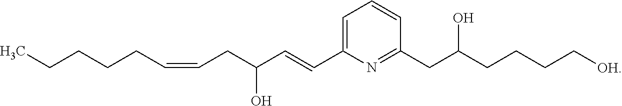 [SMILES: CCCCC/C=C\CC(O)/C=C/c1cccc(CC(O)CCCCO)n1]

In some cases, the BLT1 antagonist is CP-195494, which has the following structure:

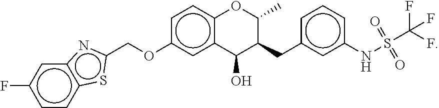

In some cases, the BLT1 antagonist is ONO-4057 (5-{2-(2-Carboxy-ethyl)-3-[(E)-6-(4-methoxy-phenyl)-hex-5-enyloxy]-phenoxy}-pentanoic acid).

In some cases, the BLT1 antagonist is montekulast, which has the following structure:

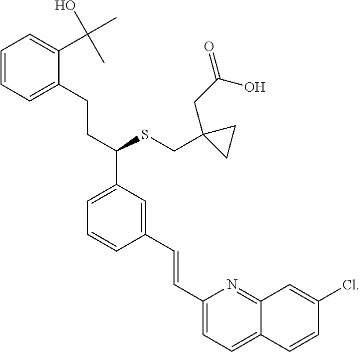 [SMILES: CC(C)(O)c1ccccc1CC[C@@H](SCC1(CC(=O)O)CC1)c1cccc(/C=C/c2ccc3ccc(Cl)cc3n2)c1]

In some cases, the BLT1 antagonist is prankulast, which has the following structure:

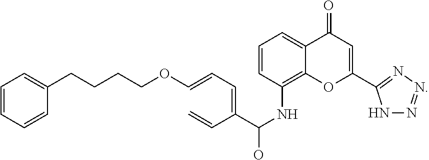 [SMILES: C=C/C(=C\C=C\OCCCCc1ccccc1)C(O)Nc1cccc2c(=O)cc(-c3nnn[nH]3)oc12]

In some cases, the BLT1 antagonist is zafirulkast, which has the following structure:

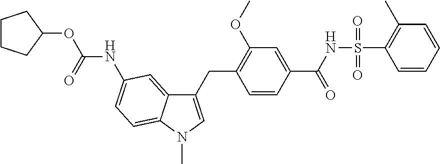 [SMILES: COc1cc(C(=O)NS(=O)(=O)c2ccccc2C)ccc1Cc1cn(C)c2ccc(NC(=O)OC3CCCC3)cc12]

In some cases, the BLT1 antagonist is selected from compounds (a) through (vv), as follows:

a) 2-[3-[3-(4-acetyl-2-ethyl-5-hydroxyphenoxy)propoxy]-2-propylphenoxy]benzoic acid;

b) (1α,3β,5Z,7E)-9,10-Secocholesta-5,7,10(19)-triene-1,3,25-triol; 1,25-Dihydroxycholecalciferol; 1,25-Dihydroxyvitamin D; 1,25-Dihydrovitamin D3; 1α,25-Dihydroxycholecalciferol; 1α,25-Dihydroxyvitamin D3;

c) (5-[[3,5-bis(1,1-dimethylethyl)-4-hydroxyphenyl]methylene]-2,4-thiazolidinedione;

d) (2-[(3S,4R)-3,4-dihyro-4-hydroxy-3-(phenylmethyl)-2H-1-benzopyran-7-yl]-4-(trifluromethyl) benzoic acid;

e) (2-fluoro-4′-(2-quinolinylmethoxy)-[1,2′-biphenyl]-4-acetic acid;

f) ((R)-α-cyclopentyl-4-(2-quinolinylmethoxy) benzeneacetic acid;

g) (4-[[5-[4-(aminoiminomethyl)phenoxy]pentyl]oxy]-3-methoxy-N,N-bis(1-methylethyl) benzamide;

h) (3 2-phenyl-1,2-benzisoselenazol-3(2H)-one;

i) (4-[[[(3-fluorophenyl)methyl] [4-(2-quinolinylmethoxy)phenyl]amino]methyl] benzoic acid;

j) (2-(4-carboxybutoxy)-6-[[6-(4-methoxyphenyl)-5-hexenyl]oxy] benzenepropanoic acid;

k) 4-[5-[[2-[4-(diphenylmethoxy)-1-piperidinyl]ethyl]amino]-5-oxo-1,3-pentadienyl]-2-methoxyphenyl ethyl ester carbonic acid;

l) ((S)—N-[2-cyclohexyl-1-(2-pyrindyl)ethyl]-5-methyl-2-benzoxazolamine; ontazolast;

m) (ONO 4057; (E)-2-(4-carboxybutoxy)-6-[[6-(4-methoxyphenyl)-5-hexenyl]oxy] benzenepropanoic acid;

n) (1-[(3S,4R0-3-([1,1′-biphenyl]-4-ylmethy)-3,4-dihydro-4-hydroxy-2H-1-benzopyran-7-yl]-Cyclopentanecarboxylic acid;

o) (1,[5-hydroxy-5-[8-(1-hydroxy-2-phenylethyl)-2-dibenzofuranyl]-1-oxopentyl] pyrroline;

p) (α,α-dimethyl-3-(3-phenylpropyl)-2-thiopheneheptanoic acid;

q) ((E)-3-[6-[[(3-aminophenyl)sulfinyl]methyl]-3-[[8-(4-methoxyphenyl)octyl]oxy]-2-pyridinyl]-2-propenoic acid;

r) ((E)-3-[[[[6-(2-carboxyethenyl)-5-[[8-(4-methoxyphenyl)octyl]oxy]-2-pyridinyl]methyl]thio]methyl] benzoic acid;

s) ((E)-3-[6-[[2,6-dichlorophenyl)thio]methyl]-3-(2-phenylethoxy-2-pyridinyl]-2-propenoic acid; ticolubant;

t) (7-[3-(2-cyclopropylmethyl)-3-methoxy-4-[(methylamino)carbonyl]phenoxy)propoxy]-3,4-dihydro-8-propyl-(S)-2H-1-benzopyran-2-propanoic acid;

u) (1-[4,11-dihydroxy-13-(4-methoxyphenyl)-1-oxo-5,7,9-tridecatrienyl] pyrrolidine;

v) (4-chloro-N-1H-1,2,4-triazol-3-yl-benzenesulfenamide;

w) (5-[[3,5-bis(1,1-dimethylethyl)-4-hydroxyphenyl]methylene]-2,4-thiazolidinedione;

x) Warner Lambert BPC-15 (CAS Registry Number 195215-25-9);

y) MacroNex MNX-160 (CAS Registry Number 195215-47-5);

z) (1-[(4-chlorophenyl)methyl]-3-[(1,1-dimethylethyl)thio]-α,α-dimethyl-5-(1-methyethyl)-1H-indole-2-propanoic acid; L 663536;

aa) Ono ONO-LB-448 (CAS Registry Number 186912-85-6);

bb) (α-pentyl-3-(2-quinolinylmethoxy) benzenemethanol;

cc) (3-[5-(4-chlorophenoxy)-3-methyl-3-pentenyl]-2-ethyl-2-methyl oxirane;

dd) (4-[2-[methyl(2-phenylethyl)amino]-2-oxoethyl]-8-(phenylmethoxy)-2-naphthalenecarboxylic acid;

ee) Rhone-Poulenc Rorer RP66364 (CAS Registry Number 186912-92-5)

ff) (2-[[5-methyl-5-(1H-tetrazol-5-yl)hexyl]oxy]-4,6-diphenyl pyridine;

gg) Shionogi S-2474 (CAS Registry Number 195215-53-3);

hh) (7-[3-[2-(cyclopropylmethyl)-3-methoxy-4-(4-thiazoly)phenoxy]propoxy]-3,4-dihydro-8-propyl-2H-1-benzopyran-2-carboxylic acid;

ii) (7-[3-(4-acetyl-3-methoxy-2-propylphenoxy)propoxy]-3,4-dihydro-8-propyl-2H-1-benzopyran-2-carboxylic acid;

jj) (7-[3-[2-(cyclopropylmethyl)-3-methoxy-4-(4-thiazolyl)phenoxy]propoxy]-3,4-dihydro-8-propyl-2H-1-benzopyran-2-carboxylic acid;

kk) (7-[3-[2-(cyclopropylmethyl)-3-methoxy-4-[(methylamino)carbonyl]phenoxy]propoxy]-3,4-dihydro-8-propyl-2H-1-benzopyran-2-propanoic acid;

ll) (7-[3-[4-(aminocarbonyl)-3-methoxy-2-propylphenoxy]propoxy]-3,4-dihydro-8-propyl-2H-1-benzopyran-2-carboxylic acid;

mm) (6,7-dihydro-2-(4-methoxyphenyl)-3-(4-pyridinyl)-5H-Pyrrolo[1,2-a]imidazole;

nn) Leo Denmark SR-2566 (CAS Registry Number 195215-55-5)

oo) Tanabe T-757 (CAS Registry 187112-56-7)

pp) [1R-[1α,2β(E)]]-(2-[[4-[2-[2-(2-naphthalenyl)ethenyl]cyclopropyl]-1-oxobutyl]amino] benzoic acid methyl ester;

qq) (5-(3-carboxybenzoyl)-2-)decyloxy) benzenepropanoic acid;

rr) (7-carboxy-3-(decyloxy)-9-oxo-9H-xanthene-4-propanoic acid;

ss) (1-[5-ethyl-2-hydroxy-4-[[6-methyl-6-(1H-tetrazol-5-yl)heptyl]oxy]phenyl] ethanone; CGS 23356;

tt) (2-ethyoxy-4-ethyl-5-[[6-methyl-6-(2H-tetrazol-5-yl)heptyl]oxy]phenol); and

uu) (3,4-dihydro-8-propyl-7-[[3-(2-ethyl-5-hydroxy-4-ethoxyphenoxy)propyl]oxy]-2H-1-benzopyran-2-carboxylic acid);

vv) Lilly LY210073 (CAS Registry Number 186912-79-8); or a pharmaceutically acceptable salt, solvate, or prodrug derivative thereof.

### Formulations, Dosages, Routes of Administration

As discussed above, a treatment method of the present disclosure generally involves administering to an individual in need thereof an effective amount of either a CXCR2 antagonist, or a BLT1 antagonist. For simplicity, the term “agent” or “active agent”, below, refers to either a CXCR2 antagonist or a BLT1 antagonist. Formulations, dosages, and routes of administration are discussed below. In some cases, a composition, e.g., a pharmaceutical composition, comprising an active compound is administered to an individual in need thereof.

In some instances, a composition comprising an active agent (a CXCR2 antagonist, or a BLT1 antagonist) can comprise a pharmaceutically acceptable excipient, a variety of which are known in the art and need not be discussed in detail herein. Pharmaceutically acceptable excipients have been amply described in a variety of publications, including, for example, A. Gennaro (1995) “Remington: The Science and Practice of Pharmacy”, 19th edition, Lippincott, Williams, & Wilkins.

For simplicity, in the following discussion of formulations, dosages, and routes of administration, a CXCR2 antagonist, or a BLT1 antagonist, as described above is referred to as an “active agent.”

### Formulations

In the subject methods, the active agent(s) may be administered to the host using any convenient means capable of resulting in the desired therapeutic effect or clinical outcome. Thus, an active agent can be incorporated into a variety of formulations for therapeutic administration. More particularly, an active agent can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, an active agent may be administered in the form of its pharmaceutically acceptable salt, or an active agent may also be used alone or in appropriate association, as well as in combination, with other pharmaceutically active compounds. The following methods and excipients are merely exemplary and are in no way limiting.

For oral preparations, an active agent can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

An active agent can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

An active agent can be utilized in aerosol formulation to be administered via inhalation. The compounds of the present invention can be formulated into pressurized acceptable propellants such as dichlorodifluoromethane, propane, nitrogen and the like.

Furthermore, an active agent can be made into suppositories by mixing with a variety of bases such as emulsifying bases or water-soluble bases. An active agent can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise an active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of an active agent calculated in an amount sufficient to produce the desired effect in association with a pharmaceutically acceptable diluent, carrier or vehicle. The specifications for a suitable dosage form depend, e.g., on the particular active agent employed and the effect to be achieved, and the pharmacodynamics associated with each compound in the host.

Other modes of administration will also find use with a subject method. For instance, an active agent can be formulated in suppositories and, in some cases, aerosol and intranasal compositions. For suppositories, the vehicle composition can include traditional binders and carriers such as, polyalkylene glycols, or triglycerides. Such suppositories may be formed from mixtures containing the active ingredient in the range of about 0.5% to about 10% (w/w), e.g., about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of an active agent by the nasal mucosa.

An active agent can be administered in a composition suitable for injection. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Oral Formulations

In some embodiments, an active agent is formulated for oral delivery to an individual in need of such an agent.

For oral delivery, a formulation comprising an active agent will in some embodiments include an enteric-soluble coating material. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™, and shellac.

As one non-limiting example of a suitable oral formulation, an active agent is formulated with one or more pharmaceutical excipients and coated with an enteric coating, as described in U.S. Pat. No. 6,346,269. For example, a solution comprising an active agent and a stabilizer is coated onto a core comprising pharmaceutically acceptable excipients, to form an active agent-coated core; a sub-coating layer is applied to the active agent-coated core, which is then coated with an enteric coating layer. The core generally includes pharmaceutically inactive components such as lactose, a starch, mannitol, sodium carboxymethyl cellulose, sodium starch glycolate, sodium chloride, potassium chloride, pigments, salts of alginic acid, talc, titanium dioxide, stearic acid, stearate, micro-crystalline cellulose, glycerin, polyethylene glycol, triethyl citrate, tributyl citrate, propanyl triacetate, dibasic calcium phosphate, tribasic sodium phosphate, calcium sulfate, cyclodextrin, and castor oil. Suitable solvents for an active agent include aqueous solvents. Suitable stabilizers include alkali-metals and alkaline earth metals, bases of phosphates and organic acid salts and organic amines. The sub-coating layer comprises one or more of an adhesive, a plasticizer, and an anti-tackiness agent. Suitable anti-tackiness agents include talc, stearic acid, stearate, sodium stearyl fumarate, glyceryl behenate, kaolin and aerosil. Suitable adhesives include polyvinyl pyrrolidone (PVP), gelatin, hydroxyethyl cellulose (HEC), hydroxypropyl cellulose (HPC), hydroxypropyl methyl cellulose (HPMC), vinyl acetate (VA), polyvinyl alcohol (PVA), methyl cellulose (MC), ethyl cellulose (EC), hydroxypropyl methyl cellulose phthalate (HPMCP), cellulose acetate phthalates (CAP), xanthan gum, alginic acid, salts of alginic acid, Eudragit™, copolymer of methyl acrylic acid/methyl methacrylate with polyvinyl acetate phthalate (PVAP). Suitable plasticizers include glycerin, polyethylene glycol, triethyl citrate, tributyl citrate, propanyl triacetate and castor oil. Suitable enteric-soluble coating material include hydroxypropyl methylcellulose acetate succinate (HPMCAS), hydroxypropyl methyl cellulose phthalate (HPMCP), cellulose acetate phthalate (CAP), polyvinyl phthalic acetate (PVPA), Eudragit™ and shellac.

Suitable oral formulations also include an active agent formulated with any of the following: microgranules (see, e.g., U.S. Pat. No. 6,458,398); biodegradable macromers (see, e.g., U.S. Pat. No. 6,703,037); biodegradable hydrogels (see, e.g., Graham and McNeill (1989) Biomaterials 5:27-36); biodegradable particulate vectors (see, e.g., U.S. Pat. No. 5,736,371); bioabsorbable lactone polymers (see, e.g., U.S. Pat. No. 5,631,015); slow release protein polymers (see, e.g., U.S. Pat. No. 6,699,504; Pelias Technologies, Inc.); a poly(lactide-co-glycolide/polyethylene glycol block copolymer (see, e.g., U.S. Pat. No. 6,630,155; Atrix Laboratories, Inc.); a composition comprising a biocompatible polymer and particles of metal cation-stabilized agent dispersed within the polymer (see, e.g., U.S. Pat. No. 6,379,701; Alkermes Controlled Therapeutics, Inc.); and microspheres (see, e.g., U.S. Pat. No. 6,303,148; Octoplus, B.V.).

Suitable oral formulations also include an active agent formulated with any of the following: a carrier such as Emisphere® (Emisphere Technologies, Inc.); TIMERx, a hydrophilic matrix combining xanthan and locust bean gums which, in the presence of dextrose, form a strong binder gel in water (Penwest); Geminex™ (Penwest); Procise™ (GlaxoSmithKline); SAVIT™ (Mistral Pharma Inc.); RingCap™ (Alza Corp.); Smartrix® (Smartrix Technologies, Inc.); SQZgel™ (MacroMed, Inc.); Geomatrix™ (Skye Pharma, Inc.); Oros® Tri-layer (Alza Corporation); and the like.

Also suitable for use are formulations such as those described in U.S. Pat. No. 6,296,842 (Alkermes Controlled Therapeutics, Inc.); U.S. Pat. No. 6,187,330 (Scios, Inc.); and the like.

Also suitable for use herein are formulations comprising an intestinal absorption enhancing agent. Suitable intestinal absorption enhancers include, but are not limited to, calcium chelators (e.g., citrate, ethylenediamine tetracetic acid); surfactants (e.g., sodium dodecyl sulfate, bile salts, palmitoylcarnitine, and sodium salts of fatty acids); toxins (e.g., zonula occludens toxin); and the like.

### Controlled Release Formulations

In some embodiments, an active agent is formulated in a controlled release formulation.

Controlled release within the scope of this invention can be taken to mean any one of a number of extended release dosage forms. The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

The various controlled release technologies cover a very broad spectrum of drug dosage forms. Controlled release technologies include, but are not limited to physical systems and chemical systems.

Physical systems include, but are not limited to, reservoir systems with rate-controlling membranes, such as microencapsulation, macroencapsulation, and membrane systems; reservoir systems without rate-controlling membranes, such as hollow fibers, ultra microporous cellulose triacetate, and porous polymeric substrates and foams; monolithic systems, including those systems physically dissolved in non-porous, polymeric, or elastomeric matrices (e.g., nonerodible, erodible, environmental agent ingression, and degradable), and materials physically dispersed in non-porous, polymeric, or elastomeric matrices (e.g., nonerodible, erodible, environmental agent ingression, and degradable); laminated structures, including reservoir layers chemically similar or dissimilar to outer control layers; and other physical methods, such as osmotic pumps, or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous). Additional discussion of categories of systems for controlled release may be found in Agis F. Kydonieus, Controlled Release Technologies: Methods, Theory and Applications, 1980 (CRC Press, Inc.).

There are a number of controlled release drug formulations that are developed for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-release gastrointestinal delivery devices; gel diffusion-controlled gastrointestinal delivery systems; and ion-exchange-controlled gastrointestinal delivery systems, which include cationic and anionic drugs. Additional information regarding controlled release drug delivery systems may be found in Yie W. Chien, Novel Drug Delivery Systems, 1992 (Marcel Dekker, Inc.). Some of these formulations will now be discussed in more detail.

Enteric coatings are applied to tablets to prevent the release of drugs in the stomach either to reduce the risk of unpleasant side effects or to maintain the stability of the drug which might otherwise be subject to degradation of expose to the gastric environment. Most polymers that are used for this purpose are polyacids that function by virtue or the fact that their solubility in aqueous medium is pH-dependent, and they require conditions with a pH higher than normally encountered in the stomach.

One exemplary type of oral controlled release structure is enteric coating of a solid or liquid dosage form. The enteric coatings are designed to disintegrate in intestinal fluid for ready absorption. Delay of absorption of the active agent that is incorporated into a formulation with an enteric coating is dependent on the rate of transfer through the gastrointestinal tract, and so the rate of gastric emptying is an important factor. In one exemplary embodiment, an active agent can be contained in an enterically coated multiple-unit dosage form. In an exemplary embodiment, a dosage form comprising an active agent is prepared by spray-coating granules of the active agent-enteric coating agent solid dispersion on an inert core material. These granules can result in prolonged absorption of the active agent with good bioavailability.

Typical enteric coating agents include, but are not limited to, hydroxypropylmethylcellulose phthalate, methacryclic acid-methacrylic acid ester copolymer, polyvinyl acetate-phthalate and cellulose acetate phthalate. Akihiko Hasegawa, Application of solid dispersions of Nifedipine with enteric coating agent to prepare a sustained-release dosage form, Chem. Pharm. Bull. 33: 1615-1619 (1985). Various enteric coating materials may be selected on the basis of testing to achieve an enteric coated dosage form designed ab initio to have an optimal combination of dissolution time, coating thicknesses and diametral crushing strength. S. C. Porter et al., The Properties of Enteric Tablet Coatings Made From Polyvinyl Acetate-phthalate and Cellulose acetate Phthalate, J. Pharm. Pharmacol. 22:42p (1970).

Another type of useful oral controlled release structure is a solid dispersion. A solid dispersion may be defined as a dispersion of one or more active ingredients in an inert carrier or matrix in the solid state prepared by the melting (fusion), solvent, or melting-solvent method. Akihiko Hasegawa, Super Saturation Mechanism of Drugs from Solid Dispersions with Enteric Coating Agents, Chem. Pharm. Bull. 36: 4941-4950 (1998). The solid dispersions may be also called solid-state dispersions. The term “coprecipitates” may also be used to refer to those preparations obtained by the solvent methods.

The selection of the carrier may have an influence on the dissolution characteristics of the dispersed active agent because the dissolution rate of a component from a surface may be affected by other components in a multiple component mixture. For example, a water-soluble carrier may result in a fast release of the drug from the matrix, or a poorly soluble or insoluble carrier may lead to a slower release of the drug from the matrix. The solubility of an active agent may also be increased owing to some interaction with the carriers.

Examples of carriers useful in solid dispersions include, but are not limited to, water-soluble polymers such as polyethylene glycol, polyvinylpyrrolidone, and hydroxypropylmethyl—cellulose. Alternative carriers include phosphatidylcholine. Phosphatidylcholine is an amphoteric but water-insoluble lipid, which may improve the solubility of otherwise insoluble active agents in an amorphous state in phosphatidylcholine solid dispersions.

Other carriers include polyoxyethylene hydrogenated castor oil. Poorly water-soluble active agents may be included in a solid dispersion system with an enteric polymer such as hydroxypropylmethylcellulose phthalate and carboxymethylethylcellulose, and a non-enteric polymer, hydroxypropylmethylcellulose. Another solid dispersion dosage form includes incorporation of an active agent with ethyl cellulose and stearic acid in different ratios.

There are various methods commonly known for preparing solid dispersions. These include, but are not limited to, the melting method, the solvent method and the melting-solvent method.

Another controlled release dosage form is a complex between an ion exchange resin and an active agent. Ion exchange resin-drug complexes have been used to formulate sustained-release products of acidic and basic drugs. In one exemplary embodiment, a polymeric film coating is provided to the ion exchange resin-drug complex particles, making drug release from these particles diffusion controlled. See Y. Raghunathan et al., Sustained-released drug delivery system I: Coded ion-exchange resin systems for phenylpropanolamine and other drugs, J. Pharm. Sciences 70: 379-384 (1981).

Injectable microspheres are another controlled release dosage form. Injectable micro spheres may be prepared by non-aqueous phase separation techniques, and spray-drying techniques. Microspheres may be prepared using polylactic acid or copoly(lactic/glycolic acid). Shigeyuki Takada, Utilization of an Amorphous Form of a Water-Soluble GPIIb/IIIa Antagonist for Controlled Release From Biodegradable Micro spheres, Pharm. Res. 14:1146-1150 (1997), and ethyl cellulose, Yoshiyuki Koida, Studies on Dissolution Mechanism of Drugs from Ethyl Cellulose Microcapsules, Chem. Pharm. Bull. 35:1538-1545 (1987).

Other controlled release technologies that may be used include, but are not limited to, SODAS (Spheroidal Oral Drug Absorption System), INDAS (Insoluble Drug Absorption System), IPDAS (Intestinal Protective Drug Absorption System), MODAS (Multiporous Oral Drug Absorption System), EFVAS (Effervescent Drug Absorption System), PRODAS (Programmable Oral Drug Absorption System), and DUREDAS (Dual Release Drug Absorption System) available from Elan Pharmaceutical Technologies. SODAS are multi particulate dosage forms utilizing controlled release beads. INDAS are a family of drug delivery technologies designed to increase the solubility of poorly soluble drugs. IPDAS are multi particulate tablet formation utilizing a combination of high density controlled release beads and an immediate-release granulate. MODAS are controlled release single unit dosage forms. Each tablet consists of an inner core surrounded by a semipermeable multiparous membrane that controls the rate of drug release. EFVAS is an effervescent drug absorption system. PRODAS is a family of multi particulate formulations utilizing combinations of immediate release and controlled release mini-tablets. DUREDAS is a bilayer tablet formulation providing dual release rates within the one dosage form. Although these dosage forms are known to one of skill, certain of these dosage forms will now be discussed in more detail.

INDAS was developed specifically to improve the solubility and absorption characteristics of poorly water soluble drugs. Solubility and, in particular, dissolution within the fluids of the gastrointestinal tract is a key factor in determining the overall oral bioavailability of poorly water soluble drug. By enhancing solubility, one can increase the overall bioavailability of a drug with resulting reductions in dosage. INDAS takes the form of a high energy matrix tablet, production of which is comprised of two distinct steps: the drug in question is converted to an amorphous form through a combination of energy, excipients, and unique processing procedures.

Once converted to the desirable physical form, the resultant high energy complex may be stabilized by an absorption process that utilizes a novel polymer cross-linked technology to prevent recrystallization. The combination of the change in the physical state of an active agent coupled with the solubilizing characteristics of the excipients employed enhances the solubility of the active agent. The resulting absorbed amorphous drug complex granulate may be formulated with a gel-forming erodible tablet system to promote substantially smooth and continuous absorption.

IPDAS is a multi-particulate tablet technology that may enhance the gastrointestinal tolerability of potential irritant and ulcerogenic drugs. Intestinal protection is facilitated by the multi-particulate nature of the IPDAS formulation which promotes dispersion of an irritant lipoate throughout the gastrointestinal tract. Controlled release characteristics of the individual beads may avoid high concentration of drug being both released locally and absorbed systemically. The combination of both approaches serves to minimize the potential harm of an active agent with resultant benefits to patients.

IPDAS is composed of numerous high density controlled release beads. Each bead may be manufactured by a two step process that involves the initial production of a micromatrix with embedded active agent and the subsequent coating of this micromatrix with polymer solutions that form a rate-limiting semipermeable membrane in vivo. Once an IPDAS tablet is ingested, it may disintegrate and liberate the beads in the stomach. These beads may subsequently pass into the duodenum and along the gastrointestinal tract, e.g., in a controlled and gradual manner, independent of the feeding state. Release of the active agent occurs by diffusion process through the micromatrix and subsequently through the pores in the rate controlling semipermeable membrane. The release rate from the IPDAS tablet may be customized to deliver a drug-specific absorption profile associated with optimized clinical benefit. Should a fast onset of activity be necessary, immediate release granulate may be included in the tablet. The tablet may be broken prior to administration, without substantially compromising drug release, if a reduced dose is required for individual titration.

MODAS is a drug delivery system that may be used to control the absorption of water soluble agents. Physically MODAS is a non-disintegrating table formulation that manipulates drug release by a process of rate limiting diffusion by a semipermeable membrane formed in vivo. The diffusion process essentially dictates the rate of presentation of drug to the gastrointestinal fluids, such that the uptake into the body is controlled. Because of the minimal use of excipients, MODAS can readily accommodate small dosage size forms. Each MODAS tablet begins as a core containing active drug plus excipients. This core is coated with a solution of insoluble polymers and soluble excipients. Once the tablet is ingested, the fluid of the gastrointestinal tract may dissolve the soluble excipients in the outer coating leaving substantially the insoluble polymer. What results is a network of tiny, narrow channels connecting fluid from the gastrointestinal tract to the inner drug core of water soluble drug. This fluid passes through these channels, into the core, dissolving the drug, and the resultant solution of drug may diffuse out in a controlled manner. This may permit both controlled dissolution and absorption. An advantage of this system is that the drug releasing pores of the tablet are distributed over substantially the entire surface of the tablet. This facilitates uniform drug absorption reduces aggressive unidirectional drug delivery. MODAS represents a very flexible dosage form in that both the inner core and the outer semipermeable membrane may be altered to suit the individual delivery requirements of a drug. In particular, the addition of excipients to the inner core may help to produce a microenvironment within the tablet that facilitates more predictable release and absorption rates. The addition of an immediate release outer coating may allow for development of combination products.

Additionally, PRODAS may be used to deliver an active agent. PRODAS is a multi particulate drug delivery technology based on the production of controlled release mini tablets in the size range of 1.5 to 4 mm in diameter. The PRODAS technology is a hybrid of multi particulate and hydrophilic matrix tablet approaches, and may incorporate, in one dosage form, the benefits of both these drug delivery systems.

In its most basic form, PRODAS involves the direct compression of an immediate release granulate to produce individual mini tablets that contain an active agent. These mini tablets are subsequently incorporated into hard gels and capsules that represent the final dosage form. A more beneficial use of this technology is in the production of controlled release formulations. In this case, the incorporation of various polymer combinations within the granulate may delay the release rate of drugs from each of the individual mini tablets. These mini tablets may subsequently be coated with controlled release polymer solutions to provide additional delayed release properties. The additional coating may be necessary in the case of highly water soluble drugs or drugs that are perhaps gastroirritants where release can be delayed until the formulation reaches more distal regions of the gastrointestinal tract. One value of PRODAS technology lies in the inherent flexibility to formulation whereby combinations of mini tablets, each with different release rates, are incorporated into one dosage form. As well as potentially permitting controlled absorption over a specific period, this also may permit targeted delivery of drug to specific sites of absorption throughout the gastrointestinal tract. Combination products also may be possible using mini tablets formulated with different active ingredients.

DUREDAS is a bilayer tableting technology that may be used to an active agent. DUREDAS was developed to provide for two different release rates, or dual release of a drug from one dosage form. The term bilayer refers to two separate direct compression events that take place during the tableting process. In an exemplary embodiment, an immediate release granulate is first compressed, being followed by the addition of a controlled release element which is then compressed onto this initial tablet. This may give rise to the characteristic bilayer seen in the final dosage form.

The controlled release properties may be provided by a combination of hydrophilic polymers. In certain cases, a rapid release of an active agent may be desirable in order to facilitate a fast onset of therapeutic affect. Hence one layer of the tablet may be formulated as an immediate release granulate. By contrast, the second layer of the tablet may release the drug in a controlled manner, e.g., through the use of hydrophilic polymers. This controlled release may result from a combination of diffusion and erosion through the hydrophilic polymer matrix.

A further extension of DUREDAS technology is the production of controlled release combination dosage forms. In this instance, two different active agents may be incorporated into the bilayer tablet and the release of drug from each layer controlled to maximize therapeutic affect of the combination.

An active agent can be incorporated into any one of the aforementioned controlled released dosage forms, or other conventional dosage forms. The amount of active agent contained in each dose can be adjusted, to meet the needs of the individual patient, and the indication. One of skill in the art and reading this disclosure will readily recognize how to adjust the level of an active agent and the release rates in a controlled release formulation, in order to optimize delivery of the active agent and its bioavailability.

### Inhalational Formulations

An active agent will in some embodiments be administered to a patient by means of a pharmaceutical delivery system for the inhalation route. An active agent may be formulated in a form suitable for administration by inhalation. The inhalational route of administration provides the advantage that the inhaled drug can bypass the blood-brain barrier. The pharmaceutical delivery system is one that is suitable for respiratory therapy by delivery of an active agent to mucosal linings of the bronchi. This invention can utilize a system that depends on the power of a compressed gas to expel an active agent from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in this invention, the aerosol contains an active agent, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Suitable propellants include, but are not limited to, hydrocarbons or other suitable gas. In the case of the pressurized aerosol, the dosage unit may be determined by providing a value to deliver a metered amount.

An active agent can also be formulated for delivery with a nebulizer, which is an instrument that generates very fine liquid particles of substantially uniform size in a gas. For example, a liquid containing an active agent is dispersed as droplets. The small droplets can be carried by a current of air through an outlet tube of the nebulizer. The resulting mist penetrates into the respiratory tract of the patient.

A powder composition containing an active agent, with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

There are several different types of inhalation methodologies which can be employed in connection with the present invention. An active agent can be formulated in basically three different types of formulations for inhalation. First, an active agent can be formulated with low boiling point propellants. Such formulations are generally administered by conventional meter dose inhalers (MDI's). However, conventional MDI's can be modified so as to increase the ability to obtain repeatable dosing by utilizing technology which measures the inspiratory volume and flow rate of the patient as discussed within U.S. Pat. Nos. 5,404,871 and 5,542,410.

Alternatively, an active agent can be formulated in aqueous or ethanolic solutions and delivered by conventional nebulizers. Lastly, an active agent can be formulated into dry powder formulations. Such formulations can be administered by simply inhaling the dry powder formulation after creating an aerosol mist of the powder.

### Dosages

Although the dosage used will vary depending on the clinical goals to be achieved, a suitable dosage range is one which provides up to about 1 μg to about 1,000 μg or about 10,000 μg of an active agent and can be administered in a single dose. Alternatively, a target dosage of an active agent can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

### Routes of Administration

An active agent is administered to an individual using any available method and route suitable for drug delivery, including in vivo and ex vivo methods, as well as systemic and localized routes of administration.

Conventional and pharmaceutically acceptable routes of administration include intranasal, intramuscular, intratracheal, intracranial, subcutaneous, intradermal, topical application, intravenous, rectal, nasal, oral and other enteral and parenteral routes of administration. Routes of administration may be combined, if desired, or adjusted depending upon the agent and/or the desired effect. The composition can be administered in a single dose or in multiple doses. In some embodiments, the composition is administered orally. In other embodiments, the composition is administered intravenously. In other embodiments, the composition is administered via an inhalational route. In other embodiments, the composition is administered intramuscularly.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes. In some cases, an active agent is administered orally. In some cases, an active agent is administered topically.

Parenteral routes of administration other than inhalation administration include, but are not necessarily limited to, topical, transdermal, subcutaneous, intramuscular, intraorbital, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as a neurological disorder and pain that may be associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), humans, and non-human primates (e.g., chimpanzees, and monkeys). In some cases, the hosts will be humans.

### Dosing

The formulation of an active agent and its subsequent administration (dosing) is within the skill of those in the art. Dosing is dependent on several criteria, including severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual active agents, and can generally be estimated based on EC50s found to be effective in vitro and in vivo animal models.

For example, a suitable dose of an active agent is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein an active agent is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

In some embodiments, multiple doses of an active agent are administered. The frequency of administration of an active agent can vary depending on any of a variety of factors, e.g., severity of the symptoms, etc. For example, in some embodiments, an active agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an active agent, e.g., the period of time over which an active agent is administered, can vary, depending on any of a variety of factors, e.g., patient response, etc. For example, an active agent can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

### Combination Therapy

A subject method of treating itch can involve administering an active agent (a CXCR2 antagonist; a BLT1 antagonist), and can further involve administering at least a second therapeutic agent. Suitable second therapeutic agents include, e.g., anti-inflammatory agents; topical or oral corticosteroids (e.g., hydrocortisone; betamethasone; fluticasone); a calcineurin inhibitor (e.g., pimecrolimus; tacrolimus); an antihistamine (e.g., diphenhydramine; hydroxyzine; etc.); cyclosporine; interferon.

### Subjects Suitable for Treatment

Subjects suitable for treatment with a subject method include individuals having acute itch and individuals having chronic itch. Subjects suitable for treatment with a subject method include individuals having itch due to an allergic reaction. Subjects suitable for treatment with a subject method include individuals having itch due to contact with urushiol. Subjects suitable for treatment with a subject method include individuals having itch due to the presence on the individual of lice. Subjects suitable for treatment with a subject method include individuals having itch due to cutaneous larva migrans. Subjects suitable for treatment with a subject method include individuals having itch due to a herpes simplex virus infection. Subjects suitable for treatment with a subject method include individuals having itch due to an insect bite. Subjects suitable for treatment with a subject method include individuals having itch due to an arachnid bit. Subjects suitable for treatment with a subject method include individuals having itch due to photodermatitis. Subjects suitable for treatment with a subject method include individuals having itch due to scabies. Subjects suitable for treatment with a subject method include individuals having itch due to urticaria. Subjects suitable for treatment with a subject method include individuals having itch due to dandruff. Subjects suitable for treatment with a subject method include individuals having itch due to punctate palmoplantar keratoderma. Subjects suitable for treatment with a subject method include individuals having itch due to psoriasis. Subjects suitable for treatment with a subject method include individuals having itch due to eczema. Subjects suitable for treatment with a subject method include individuals having itch due to sunburn. Subjects suitable for treatment with a subject method include individuals having itch due to athlete's foot (fungal infection). Subjects suitable for treatment with a subject method include individuals having itch due to hidradenitis suppurativa. Subjects suitable for treatment with a subject method include individuals having itch due to xerosis. Subjects suitable for treatment with a subject method include individuals having itch due to uremic pruritis. Subjects suitable for treatment with a subject method include individuals having itch due to polycythemia. Subjects suitable for treatment with a subject method include individuals having itch due to dermatitis. Subjects suitable for treatment with a subject method include individuals having itch due to atopic dermatitis.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-11 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of treating itch in an individual, the method comprising administering to the individual an effective amount of a chemokine (C-X-C motif) receptor 2 (CXCR2) antagonist or a leuktotriene B4 receptor (BLT1) antagonist.

Aspect 2. The method of aspect 1, wherein the itch is chronic itch.

Aspect 3. The method of aspect 1, wherein the itch is due to psoriasis, eczema, or atopic dermatitis.

Aspect 4. The method of aspect 1, comprising administering the individual an effective amount of a CXCR2 antagonist.

Aspect 5. The method of aspect 1, comprising administering the individual an effective amount of a BLT1 antagonist.

Aspect 6. The method of any one of aspects 1-5, wherein the CXCR2 antagonist or the BLT1 antagonist is administered subcutaneously.

Aspect 7. The method of any one of aspects 1-5, wherein the CXCR2 antagonist or the BLT1 antagonist is administered intradermally.

Aspect 8. The method of any one of aspects 1-7, wherein the individual is a human.

Aspect 9. The method of any one of aspects 1-4 and 6-8, wherein the CXCR2 antagonist is selected from the group consisting of: MK-7123 (navarixin); danirixin; AZD5069; reparixin; CX4338; AZD-4721; SB-33235; elubrixin; PS-291822; SB-225002; SX-517; and NSC 157449.

Aspect 10. The method of any one of aspects 1-3 and 5-8, wherein the BLT1 antagonist selected from the group consisting of: pranlukast hydrate, montelukast, zafirlukast, MCC-847, KCA-757, CS-615, YM-158, L-740515, CP-195494, LM-1484, RS-635, A-93178, S-36496, BIIL-284, ONO-4057, BIIL-260, and BIIL-315.

Aspect 11. The method of any one of aspects 1-3 and 5-8, wherein the BLT1 antagonist selected from the group consisting of:

a) 2-[3-[3-(4-acetyl-2-ethyl-5-hydroxyphenoxy)propoxy]-2-propylphenoxy]benzoic acid;

b) (1α,β,5Z,7E)-9,10-Secocholesta-5,7,10(19)-triene-1,3,25-triol; 1,25-Dihydroxycholecalciferol; 1,25-Dihydroxyvitamin D; 1,25-Dihydrovitamin D3; 1α,25-Dihydroxycholecalciferol; 1α,25-Dihydroxyvitamin D3;

c) (5-[[3,5-bis(1,1-dimethylethyl)-4-hydroxyphenyl]methylene]-2,4-thiazolidinedione;

d) (2-[(3S,4R)-3,4-dihyro-4-hydroxy-3-(phenylmethyl)-2H-1-benzopyran-7-yl]-4-(trifluromethyl) benzoic acid;

e) (2-fluoro-4′-(2-quinolinylmethoxy)-[1,2′-biphenyl]-4-acetic acid;

f) ((R)-α-cyclopentyl-4-(2-quinolinylmethoxy) benzeneacetic acid;

g) (4-[[5-[4-(aminoiminomethyl)phenoxy]pentyl]oxy]-3-methoxy-N,N-bis(1-methylethyl) benzamide;

h) (3 2-phenyl-1,2-benzisoselenazol-3(2H)-one;

i) (4-[[[(3-fluorophenyl)methyl] [4-(2-quinolinylmethoxy)phenyl]amino]methyl] benzoic acid;

j) (2-(4-carboxybutoxy)-6-[[6-(4-methoxyphenyl)-5-hexenyl]oxy] benzenepropanoic acid;

k) 4-[5-[[2-[4-(diphenylmethoxy)-1-piperidinyl]ethyl]amino]-5-oxo-1,3-pentadienyl]-2-methoxyphenyl ethyl ester carbonic acid;

l) ((S)—N-[2-cyclohexyl-1-(2-pyrindyl)ethyl]-5-methyl-2-benzoxazolamine; ontazolast;

m) (ONO 4057; (E)-2-(4-carboxybutoxy)-6-[[6-(4-methoxyphenyl)-5-hexenyl]oxy] benzenepropanoic acid;

n) (1-[(3S,4R0-3-([1,1′-biphenyl]-4-ylmethy)-3,4-dihydro-4-hydroxy-2H-1-benzopyran-7-yl]-Cyclopentanecarboxylic acid;

o) (1,[5-hydroxy-5-[8-(1-hydroxy-2-phenylethyl)-2-dibenzofuranyl]-1-oxopentyl] pyrroline;

p) (α,α-dimethyl-3-(3-phenylpropyl)-2-thiopheneheptanoic acid;

q) ((E)-3-[6-[[(3-aminophenyl)sulfinyl]methyl]-3-[[8-(4-methoxyphenyl)octyl]oxy]-2-pyridinyl]-2-propenoic acid;

r) ((E)-3-[[[[6-(2-carboxyethenyl)-5-[[8-(4-methoxyphenyl)octyl]oxy]-2-pyridinyl]methyl]thio]methyl] benzoic acid;

s) ((E)-3-[6-[[2,6-dichlorophenyl)thio]methyl]-3-(2-phenylethoxy-2-pyridinyl]-2-propenoic acid; ticolubant;

t) (7-[3-(2-cyclopropylmethyl)-3-methoxy-4-[(methylamino)carbonyl]phenoxy)propoxy]-3,4-dihydro-8-propyl-(S)-2H-1-benzopyran-2-propanoic acid;

u) (1-[4,11-dihydroxy-13-(4-methoxyphenyl)-1-oxo-5,7,9-tridecatrienyl] pyrrolidine;

v) (4-chloro-N-1H-1,2,4-triazol-3-yl-benzenesulfenamide;

w) (5-[[3,5-bis(1,1-dimethylethyl)-4-hydroxyphenyl]methylene]-2,4-thiazolidinedione;

x) Warner Lambert BPC-15 (CAS Registry Number 195215-25-9);

y) MacroNex MNX-160 (CAS Registry Number 195215-47-5);

z) (1-[(4-chlorophenyl)methyl]-3-[(1,1-dimethylethyl)thio]-α,α-dimethyl-5-(1-methyethyl)-1H-indole-2-propanoic acid; L 663536;

aa) Ono ONO-LB-448 (CAS Registry Number 186912-85-6);

bb) (α-pentyl-3-(2-quinolinylmethoxy) benzenemethanol;

cc) (3-[5-(4-chlorophenoxy)-3-methyl-3-pentenyl]-2-ethyl-2-methyl oxirane;

dd) (4-[2-[methyl(2-phenylethyl)amino]-2-oxoethyl]-8-(phenylmethoxy)-2-naphthalenecarboxylic acid;

ee) Rhone-Poulenc Rorer RP66364 (CAS Registry Number 186912-92-5)

ff) (2-[[5-methyl-5-(1H-tetrazol-5-yl)hexyl]oxy]-4,6-diphenyl pyridine;

gg) Shionogi S-2474 (CAS Registry Number 195215-53-3);

hh) (7-[3-[2-(cyclopropylmethyl)-3-methoxy-4-(4-thiazoly)phenoxy]propoxy]-3,4-dihydro-8-propyl-2H-1-benzopyran-2-carboxylic acid;

ii) (7-[3-(4-acetyl-3-methoxy-2-propylphenoxy)propoxy]-3,4-dihydro-8-propyl-2H-1-benzopyran-2-carboxylic acid;

jj) (7-[3-[2-(cyclopropylmethyl)-3-methoxy-4-(4-thiazolyl)phenoxy]propoxy]-3,4-dihydro-8-propyl-2H-1-benzopyran-2-carboxylic acid;

kk) (7-[3-[2-(cyclopropylmethyl)-3-methoxy-4-[(methylamino)carbonyl]phenoxy]propoxy]-3,4-dihydro-8-propyl-2H-1-benzopyran-2-propanoic acid;

ll) (7-[3-[4-(aminocarbonyl)-3-methoxy-2-propylphenoxy]propoxy]-3,4-dihydro-8-propyl-2H-1-benzopyran-2-carboxylic acid;

mm) (6,7-dihydro-2-(4-methoxyphenyl)-3-(4-pyridinyl)-5H-Pyrrolo[1,2-a]imidazole;

nn) Leo Denmark SR-2566 (CAS Registry Number 195215-55-5)

oo) Tanabe T-757 (CAS Registry 187112-56-7)

pp) [1R-[1α,2β(E)]]-(2-[[4-[2-[2-(2-naphthalenyl)ethenyl]cyclopropyl]-1-oxobutyl]amino] benzoic acid methyl ester;

qq) (5-(3-carboxybenzoyl)-2-)decyloxy) benzenepropanoic acid;

rr) (7-carboxy-3-(decyloxy)-9-oxo-9H-xanthene-4-propanoic acid;

ss) (1-[5-ethyl-2-hydroxy-4-[[6-methyl-6-(1H-tetrazol-5-yl)heptyl]oxy]phenyl] ethanone; CGS 23356;

tt) (2-ethyoxy-4-ethyl-5-[[6-methyl-6-(2H-tetrazol-5-yl)heptyl]oxy]phenol); and

uu) (3,4-dihydro-8-propyl-7-[[3-(2-ethyl-5-hydroxy-4-ethoxyphenoxy)propyl]oxy]-2H-1-benzopyran-2-carboxylic acid); and

vv) Lilly LY210073 (CAS Registry Number 186912-79-8).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

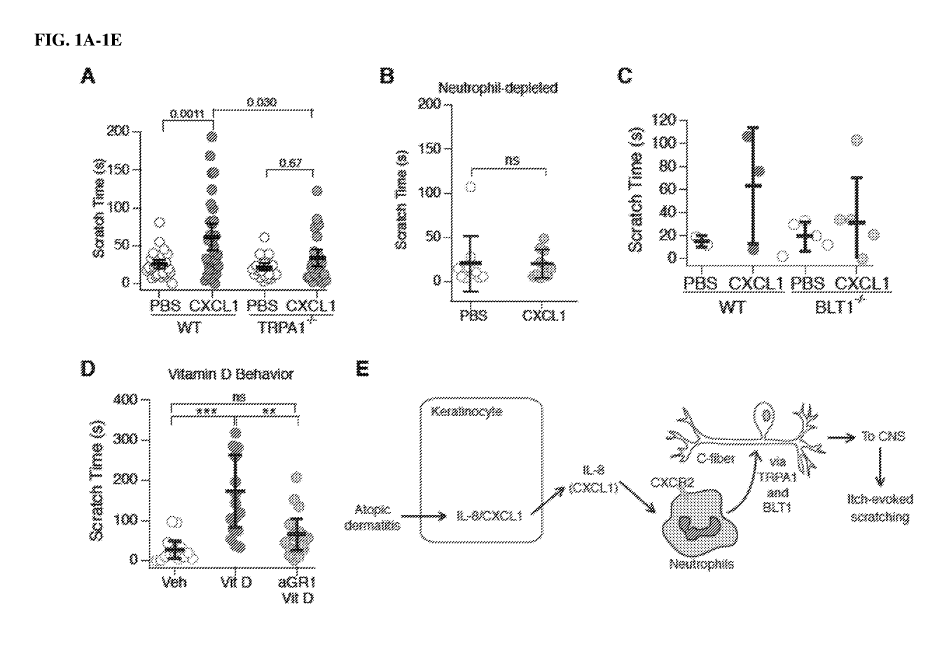

Here it has been demonstrated that CXCL1, a neutrophil chemoattractant and natural ligand for CXCR2, robustly induces itch when injected subcutaneously in mice (FIG. 1A). CXCL1 is the mouse homologue for human Interleukin-8, which is increased in several inflammatory skin disorders including psoriasis and atopic dermatitis. Here it has also been demonstrated that IL8 is increased in primary human keratinocytes upon activation by multiple itch agonists. Importantly, CXCL1-induced itch is entirely dependent on neutrophils (FIG. 1B). Further, mice lacking TRPA1 show reduced itch responses to CXCL1 (FIG. 1A), and TRPA1 has previously been implicated in several forms of histamine-independent acute and chronic itch. In addition, loss of TRPA1 results in reduced CXCL1 expression in skin. Blocking/ablation of the high-affinity LTB4 receptor, BLT1, also results in reduction of CXCL1-induced itch (FIG. 1C). Both LTB4 and reactive oxygen species (ROS) are produced by neutrophils and have been proposed to directly activate neurons and induce itch. It is shown here that neutrophils also play a key role in chronic itch. In a mouse model of atopic dermatitis, depletion of neutrophils during induction of itch drastically reduces itch and inflammatory responses (FIG. 1D). It is shown here that CXCL1 is increased in multiple mouse models of chronic itch, and LTB4 is increased in skin of mice with atopic dermatitis. It is proposed that neutrophils activate sensory neurons through the ion channel TRPA1 in a CXCL1/BLT1/LTB4-dependent fashion (FIG. 1E).

FIG. 1A-1E. (A) Injection of CXCL1 induces robust itch in mice, which is dependent on expression of TRPA1. (B) Depletion of neutrophils abolishes CXCL1-induced itch. (C) Mice lacking the LTB4 receptor (BLT1) show reduced itch responses to CXCL1 injection. (D) Depletion of neutrophils in a mouse model of atopic dermatitis greatly reduces itch. (E) Model showing proposed interactions. Atopic dermatitis results in increased levels of IL8 (humans) or CXCL1 (mice) that recruits neutrophils. Neutrophils activate sensory neurons to promote itch in a TRPA1 and BLT1-dependent manner. For all data, one-way ANOVA was performed with a Tukey-Kramer post hoc statistical analysis. Tukey-Kramer values are reported and only p<0.05 were considered significant; **=p<0.005; ***=p<0.0005; n.s.=not significant.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating itch in an individual, the method comprising administering to the individual an effective amount of a chemokine (C-X-C motif) receptor 2 (CXCR2) antagonist or a leuktotriene B4 receptor (BLT1) antagonist.

2. The method of claim 1, wherein the itch is chronic itch.

3. The method of claim 1, wherein the itch is due to psoriasis, eczema, or atopic dermatitis.

4. The method of claim 1, comprising administering the individual an effective amount of a CXCR2 antagonist.

5. The method of claim 1, comprising administering the individual an effective amount of a BLT1 antagonist.

6. The method of any one of claims 1-5, wherein the CXCR2 antagonist or the BLT1 antagonist is administered subcutaneously.

7. The method of any one of claims 1-5, wherein the CXCR2 antagonist or the BLT1 antagonist is administered intradermally.

8. The method of any one of claims 1-7, wherein the individual is a human.

9. The method of any one of claims 1-4 and 6-8, wherein the CXCR2 antagonist is selected from the group consisting of: MK-7123 (navarixin); danirixin; AZD5069; reparixin; CX4338; AZD-4721; SB-33235; elubrixin; PS-291822; SB-225002; SX-517; and NSC 157449.

10. The method of any one of claims 1-3 and 5-8, wherein the BLT1 antagonist selected from the group consisting of: pranlukast hydrate, montelukast, zafirlukast, MCC-847, KCA-757, CS-615, YM-158, L-740515, CP-195494, LM-1484, RS-635, A-93178, S-36496, BIIL-284, ONO-4057, BIIL-260, and BIIL-315.

11. The method of any one of claims 1-3 and 5-8, wherein the BLT1 antagonist selected from the group consisting of:
a) 2-[3-[3-(4-acetyl-2-ethyl-5-hydroxyphenoxy)propoxy]-2-propylphenoxy]benzoic acid;
b) (1α,β,5Z,7E)-9,10-Secocholesta-5,7,10(19)-triene-1,3,25-triol; 1,25-Dihydroxycholecalciferol; 1,25-Dihydroxyvitamin D; 1,25-Dihydrovitamin D3; 1α,25-Dihydroxycholecalciferol; 1α,25-Dihydroxyvitamin D3;
c) (5-[[3,5-bis(1,1-dimethylethyl)-4-hydroxyphenyl]methylene]-2,4-thiazolidinedione;
d) (2-[(3S,4R)-3,4-dihydro-4-hydroxy-3-(phenylmethyl)-2H-1-benzopyran-7-yl]-4-(trifluromethyl) benzoic acid;
e) (2-fluoro-4′-(2-quinolinylmethoxy)-[1,2′-biphenyl]-4-acetic acid;
f) ((R)-α-cyclopentyl-4-(2-quinolinylmethoxy) benzeneacetic acid;
g) (4-[[5-[4-(aminoiminomethyl)phenoxy]pentyl]oxy]-3-methoxy-N,N-bis(1-methylethyl) benzamide;
h) (3 2-phenyl-1,2-benzisoselenazol-3(2H)-one;
i) (4-[[[(3-fluorophenyl)methyl] [4-(2-quinolinylmethoxy)phenyl]amino]methyl] benzoic acid;
j) (2-(4-carboxybutoxy)-6-[[6-(4-methoxyphenyl)-5-hexenyl]oxy] benzenepropanoic acid;
k) 4-[5-[[2-[4-(diphenylmethoxy)-1-piperidinyl]ethyl]amino]-5-oxo-1,3-pentadienyl]-2-methoxyphenyl ethyl ester carbonic acid;
l) ((S)—N-[2-cyclohexyl-1-(2-pyrindyl)ethyl]-5-methyl-2-benzoxazolamine; ontazolast;
m) (ONO 4057; (E)-2-(4-carboxybutoxy)-6-[[6-(4-methoxyphenyl)-5-hexenyl]oxy] benzenepropanoic acid;
n) (1-[(3S,4R0-3-([1,1′-biphenyl]-4-ylmethy)-3,4-dihydro-4-hydroxy-2H-1-benzopyran-7-yl]-Cyclopentanecarboxylic acid;
o) (1,[5-hydroxy-5-[8-(1-hydroxy-2-phenylethyl)-2-dibenzofuranyl]-1-oxopentyl] pyrroline;
p) (α,α-dimethyl-3-(3-phenylpropyl)-2-thiopheneheptanoic acid;
q) ((E)-3-[6-[[(3-aminophenyl)sulfinyl]methyl]-3-[[8-(4-methoxyphenyl)octyl]oxy]-2-pyridinyl]-2-propenoic acid;
r) ((E)-3-[[[[6-(2-carboxyethenyl)-5-[[8-(4-methoxyphenyl)octyl]oxy]-2-pyridinyl]methyl]thio]methyl] benzoic acid;
s) ((E)-3-[6-[[2,6-dichlorophenyl)thio]methyl]-3-(2-phenylethoxy-2-pyridinyl]-2-propenoic acid; ticolubant;
t) (7-[3-(2-cyclopropylmethyl)-3-methoxy-4-[(methylamino)carbonyl]phenoxy)propoxy]-3,4-dihydro-8-propyl-(S)-2H-1-benzopyran-2-propanoic acid;
u) (1-[4,11-dihydroxy-13-(4-methoxyphenyl)-1-oxo-5,7,9-tridecatrienyl] pyrrolidine;
v) (4-chloro-N-1H-1,2,4-triazol-3-yl-benzenesulfenamide;
w) (5-[[3,5-bis(1,1-dimethylethyl)-4-hydroxyphenyl]methylene]-2,4-thiazolidinedione;
x) Warner Lambert BPC-15 (CAS Registry Number 195215-25-9);
y) MacroNex MNX-160 (CAS Registry Number 195215-47-5);
z) (1-[(4-chlorophenyl)methyl]-3-[(1,1-dimethylethyl)thio]-α,α-dimethyl-5-(1-methyethyl)-1H-indole-2-propanoic acid; L 663536;
aa) Ono ONO-LB-448 (CAS Registry Number 186912-85-6);
bb) (α-pentyl-3-(2-quinolinylmethoxy) benzenemethanol;
cc) (3-[5-(4-chlorophenoxy)-3-methyl-3-pentenyl]-2-ethyl-2-methyl oxirane;
dd) (4-[2-[methyl(2-phenylethyl)amino]-2-oxoethyl]-8-(phenylmethoxy)-2-naphthalenecarboxylic acid;
ee) Rhone-Poulenc Rorer RP66364 (CAS Registry Number 186912-92-5)
ff) (2-[[5-methyl-5-(1H-tetrazol-5-yl)hexyl]oxy]-4,6-diphenyl pyridine;
gg) Shionogi S-2474 (CAS Registry Number 195215-53-3);
hh) (7-[3-[2-(cyclopropylmethyl)-3-methoxy-4-(4-thiazoly)phenoxy]propoxy]-3,4-dihydro-8-propyl-2H-1-benzopyran-2-carboxylic acid;
ii) (7-[3-(4-acetyl-3-methoxy-2-propylphenoxy)propoxy]-3,4-dihydro-8-propyl-2H-1-benzopyran-2-carboxylic acid;
jj) (7-[3-[2-(cyclopropylmethyl)-3-methoxy-4-(4-thiazolyl)phenoxy]propoxy]-3,4-dihydro-8-propyl-2H-1-benzopyran-2-carboxylic acid;
kk) (7-[3-[2-(cyclopropylmethyl)-3-methoxy-4-[(methylamino)carbonyl]phenoxy]propoxy]-3,4-dihydro-8-propyl-2H-1-benzopyran-2-propanoic acid;
ll) (7-[3-[4-(aminocarbonyl)-3-methoxy-2-propylphenoxy]propoxy]-3,4-dihydro-8-propyl-2H-1-benzopyran-2-carboxylic acid;
mm) (6,7-dihydro-2-(4-methoxyphenyl)-3-(4-pyridinyl)-5H-Pyrrolo[1,2-a]imidazole;
nn) Leo Denmark SR-2566 (CAS Registry Number 195215-55-5)
oo) Tanabe T-757 (CAS Registry 187112-56-7)
pp) [1R-[1α,2β(E)]]-(2-[[4-[2-[2-(2-naphthalenyl)ethenyl]cyclopropyl]-1-oxobutyl]amino] benzoic acid methyl ester;
qq) (5-(3-carboxybenzoyl)-2-)decyloxy) benzenepropanoic acid;
rr) (7-carboxy-3-(decyloxy)-9-oxo-9H-xanthene-4-propanoic acid;
ss) (1-[5-ethyl-2-hydroxy-4-[[6-methyl-6-(1H-tetrazol-5-yl)heptyl]oxy]phenyl] ethanone; CGS 23356;
tt) (2-ethyoxy-4-ethyl-5-[[6-methyl-6-(2H-tetrazol-5-yl)heptyl]oxy]phenol); and
uu) (3,4-dihydro-8-propyl-7-[[3-(2-ethyl-5-hydroxy-4-ethoxyphenoxy)propyl]oxy]-2H-1-benzopyran-2-carboxylic acid); and
vv) Lilly LY210073 (CAS Registry Number 186912-79-8).
